(19) United States
(12) Patent Application Publication
(10) Pub. No.: US 2004/0157209 A1
(43) Pub. Date: Aug. 12, 2004

(54) Molecular imprinting

(76) Inventors: Ecevit Yilmaz, Bjarred (SE); Karsten Haupt, Paris (FR); Klaus Mosbach, Furulund (SE); Domingo Sanchez, Tollered (SE); Per Moller, Vallda (SE)

Correspondence Address: BURNS DOANE SWECKER & MATHIS L L P, POST OFFICE BOX 1404, ALEXANDRIA, VA, 22313-1404, US

(21) Appl. No.: 10/296,154

(22) PCT Filed: May 21, 2001
(86) PCT No.: PCT/SE01/01128

(30) Foreign Application Priority Data
May 22, 2000 (SE) 0001877-0

(51) Int. Cl. C12Q 1/70; C12Q 1/68; G01N 33/53; C12N 11/08; C12M 1/34

(52) U.S. Cl. 435/5; 435/6; 435/7.1; 435/287.2

(57) ABSTRACT

The present invention relates to molecularly imprinted polymers comprising tailor-made recognition sites for a target in which said recognition sites are located at or close to the surface of the polymer and/or of pores in the polymer. The molecularly imprinted polymer comprising tailor-made recognition sites for a target is obtainable by polymerising functional monomers and, optionally, cross-linked, optionally in a reaction solvent, in the presence of at least one template immobilised on a support material in a polymerisation process, whereby non-covalent or covalent are formed between said functional monomers and said immobilised template(s), and removing said template(s), and said support material from the molecularly imprinted polymer.

### TECHNICAL FIELD OF THE INVENTION

The present invention relates to molecularly imprinted polymers comprising tailor-made recognition sites, to a method of preparing the same, and to different applications of said molecularly imprinted polymers.

### BACKGROUND ART OF THE INVENTION

Molecular imprinting is a technique for the preparation of synthetic polymers containing recognition sites for certain target molecules [1]. This is achieved by co-polymerising functional and cross-linking monomers in the presence of the target molecule, which acts as a molecular template. The functional monomers arrange specifically around the molecular template, and are subsequently held in position by polymerisation with a usually high degree of cross-linking. After polymerisation the molecular template is extracted from the polymer, revealing complementary binding sites that allow rebinding of the target molecule with in many cases very high specificity, comparable to that of antibodies [2,3] (FIG. 1). The so obtained artificial receptors have been used in different applications that require specific ligand binding, such as separation of closely related compounds [4] and immunoassay-type binding assays [2,5]. Another application has been as recognition elements in chemical or biosensors [6-8].

Molecularly imprinted polymers, hereafter referred to as MIPs, have been produced that specifically recognise herbicides [9,10], drugs [2,5], hormones [3,11] and many other compounds including proteins [1]. Polymers can be imprinted with substances for which natural receptors do not exist or are difficult to obtain. Moreover, imprinted polymers can be used in organic solvents, and because of their great chemical, thermal and mechanical stability, they retain their molecular memory over long time periods and in harsh environments. They may therefore have considerable advantages over biomolecules as recognition elements in many applications.

There are two distinct imprinting approaches, namely non-covalent and covalent imprinting. Covalent imprinting protocols are based on covalent interactions between template and functional monomers. Examples of such systems are the use of polymerisable boronate compounds (e.g. vinylphenyl boronic acid) which form reversible covalent bonds with vicinal diols of the target molecule. After polymer formation the template is removed by chemical cleavage, leaving behind a specific binding site. Rebinding of the target molecule to the MIP is again based on reversible covalent bonds [12]. Non-covalent molecular imprinting relies on non-covalent interactions, such as hydrogen bonds, ionic bonds, pi-pi stacking or hydrophobic interactions, between the template and functional monomers. After polymer formation, the template can be removed from the MIP simply by solvent extraction. Rebinding of the target molecule is again via non-covalent interactions. One example of this approach is the imprinting of amino acid derivatives using methacrylic acid and 4-vinylpyridine as functional monomers [13]. The two approaches can be combined if the imprinting is performed using covalent bonds between template and functional monomers, whereas upon usage of the MIP, the target molecule rebinds via non-covalent interactions [14].

Molecular imprinting has until now only been performed with the template free in solution. Resulting drawbacks are a certain heterogeneity of the binding sites in the MIP regarding their orientation, shape, and their affinity and accessibility for the target molecule, which are mainly due to the high degree of freedom of the template during the imprinting process. In contrast, natural binders such as, enzymes, antibodies, receptors or nucleic acids, usually have ligand binding sites that are uniform, structurally well defined, and oriented. The development of a molecular imprinting method resulting in binding sites with characteristics that more closely resemble those of natural binders is therefore highly desirable.

### SUMMARY OF THE INVENTION

The object of the present invention is therefore to provide molecularly imprinted polymers in which the binding sites are uniform, structurally well defined and oriented. Another object of the invention is to provide molecularly imprinted polymers with binding sites having characteristics that more closely resemble those of natural binders, as well as being more accessible for targets.

These objects are obtained according to the invention by the novel molecularly imprinted polymers in which the binding sites for a target are located at or close to the surface of the polymer and/or of pores in the polymer.

The novel molecularly imprinted polymers according to the invention are obtainable by polymerising functional monomers and, optionally, cross-linkers, optionally in a reaction solvent, in the presence of at least one template immobilised on a support material in a polymerisation process, whereby non-covalent or covalent interactions are formed between said functional monomers and said immobilised template(s), and removing said template(s) and said support material from the molecularly imprinted polymer.

There are several advantages in using immobilised templates for imprinting. Templates that are not soluble in the polymerisation cocktail can be immobilised and then brought into contact with the monomers. Aggregation of the templates in the pre-polymerisation mixture, which may result in heterogeneous binding sites, car be prevented by immobilising the template to an appropriate support material. Oriented immobilisation of the template prior to polymerisation results in a uniform orientation of the binding sites, and their homogeneity is increased as immobilisation reduces the tumbling rate of the template during the imprinting polymerisation step. Another advantage in using immobilised templates eliminates the need of a porogenic solvent, the porous structure of the polymer being created after removal of the support material. As a consequence, all binding sites are therefore located at or close to the surface of the pores, resulting in a greatly improved accessibility of the sites for the analyte.

The present imprinting technique also opens new possibilities for the application of MIPs. For example, the target can be tagged with various markers, whereby the problem of sterical hindrance, which is most often encountered with classical MIPs where the binding sites are buried in the polymer structure, is avoided. This greatly extends the usefulness of MIPs for example in immunoassays and sensors, as separation materials and in chemical synthesis.

### DETAILED DESCRIPTION OF THE INVENTION

The molecularly imprinted polymers (MIPs) according to the invention comprise tailor-made recognition sites for a target, in which said recognition sites are located at or close to the surface of the polymer and/or of pores in the polymer. In one aspect of the invention said recognition sites are specific binding sites.

The molecularly imprinted polymers according to the invention are obtainable by polymerising functional monomers and, optionally, cross-linkers, optionally in a reaction solvent, in the presence of at least one template immobilised on a support material in a polymerisation process, whereby non-covalent or covalent interactions are formed between said functional monomers and said immobilised template(s); and removing said template(s) and said support material from the molecularly imprinted polymer.

In one aspect the MIPs are obtained by using a template, used in the imprinting process, and a target, for the specific rebinding to the MIPs, wherein said template and target, respectively, are the same. In another aspect the MIPs are obtained by using a template, used in the imprinting process, and a target, for the specific rebinding to the MIPs, wherein said template and said target are different.

In yet another aspect the MIPs are obtained in a process, wherein the immobilised template used in the imprinting process is a shape-forming template. In another aspect the MIPs are obtained, wherein the immobilised template used in the imprinting process is a transition state or product analogue of one or more entities of a reaction.

In the present invention there is also provided a method for preparing a molecularly imprinted polymer comprising tailor-made recognition sites for a target, which method comprises: polymerising functional monomers and, optionally, cross-linkers, optionally in a reaction solvent, in the presence of at least one template immobilised on a support material in a polymerisation process, whereby non-covalent or covalent interactions are formed between said functional monomers and said immobilised template(s), and removing said template(s) and said support material from the molecularly imprinted polymer.

The immobilised template used in the method can take the same form as outlined above when referring to different aspects of the MIPs according to the invention.

The template used in the method as well as the target for the rebinding can take the same form as outlined above when referring to different aspects of the MIPs according to the invention.

The support material used in the method can be present either in an insoluble or a colloidal form. The support material for the immobilisation of the template is then selected from the group comprising glass, silica, latex beads, polysaccharides, chitosan, ceramics, gold, agarose, other organic and inorganic macromolecular and polymeric materials, and derivatives thereof. The support material used in the method can also be present in a soluble form. Then, the support material for the immobilisation of the template is selected from the group comprising soluble polymers such as polyethyleneglycol, polyvinylalcohol, polyamide, polyester, other organic and inorganic macromolecular and polymeric materials, and derivatives thereof.

In one embodiment of the method the MIPs are prepared with immobilised templates by chemical coupling of the template or a template derivative onto solid, colloidal or soluble support materials, or by physical or chemical adhesion to solid, colloidal or soluble support materials, followed by the synthesis of the MIP in the presence of the resulting template-support material complex, and subsequent removal of the support material and the template by chemical dissolution, solvent extraction, acid or base extraction, heat, ultrasonication, mechanical or other means (Examples 1-6). In another embodiment of the method, said template(s) is/are immobilised directly onto said support material or adsorbed onto or inserted into a self-assembled monolayer preformed on said support material.

According to one aspect the template is immobilised to the support material by chemical coupling or chemical or physical adsorption, then the MIP is prepared using the template-support material complex, after which the support material is sacrificed and the template removed from the polymer. The support material and the template can be removed at the same time or in two steps, wherein, in the latter case, said support material is removed in one step and said template(s) in another. The support material may be functionalised, derivatised or activated. Said removing of the immobilised support material and the template(s) may be performed by chemical dissolution, solvent extraction, heat, ultrasonication, acid or base extraction, mechanical or other means.

In the method the total volume of the polymerisable monomer/crosslinker is up to 100%. It may also be present very diluted (i.e. 0.01%) in a solvent. The reaction solvent is either aqueous or non-aqueous, and is either composed of a single solvent component or multiple solvent components.

The polymerisation of monomers and crosslinkers in the method may be initiated by heat, by UV, by γ radiation, by visible light or by chemical means. The polymerisation process may be a free radical, an ionic, a coordination, a step growth, a living polymerisation process or another polymerisation process. The monomers used in the polymerisation process can either have the same or different functionalities.

The expression “target” that is used throughout the present application, when referring to the MIPs, the method for preparing the MIPs and the applications, is meant to be any kind of entity capable of rebinding to the MIPs according to the invention. Said target may be chosen from the group comprising a pesticide, drug, hormone, enzyme, antibody, receptor, nucleic acid, virus, cell, tissue and any other material including proteins.

The expression “template” that is used throughout the present application, when referring to the MIPs, the method for preparing the MIPs and the applications, is meant to be any kind of entity capable of being used in the imprinting process for preparing the MIPs according to the invention. Said template may be chosen from the group comprising a pesticide, drug, hormone, enzyme, antibody, receptor, nucleic acid, virus, cell, tissue and any other material including proteins.

Non-limiting applications of the MIPs prepared with immobilised templates according to the invention are as artificial receptors in applications based on specific binding. The MIPs may also be used as recognition elements in competitive or direct immunoassay-like binding assays for recognising a pesticide, drug, hormone, enzyme, antibody receptor, nucleic acid, virus, cell, tissue and other compounds including proteins. Other applications of the MIPs are as tailor-made separation and/or extraction materials, and as enzyme-like or chemical catalysts in chemical synthesis or as solid-phase extraction materials for assisted synthesis (Examples 7-10). The MIPs according to the invention may also be used as recognition element in a chemical or biosensor, as well as being used as stationary phase or soluble selector in capillary electrophoresis, capillary electrochromatography, HPLC analysis, preparative HPLC or chromatography in general.

In applications using, for detection, competitive binding, the target can be tagged with a marker such as an enzyme, a fluorescent, electrochemical, electroluminescent or magnetic label, a radioisotope, a dye, a colloidal gold particle, or another suitable entity.

In conclusion we have demonstrated here for the first time that imprinting of immobilised templates is feasible and that the MIPs thereof have many advantages to classical MIPs.

The following examples describe the preparation of the MIPs and different applications of the MIPs. The intention of the examples is illustrative only and is not to be construed as limiting in any way of the scope of the protection.

### BRIEF DESCRIPTION OF THE DRAWINGS

FIG. 1 depicts the principle of molecular imprinting.

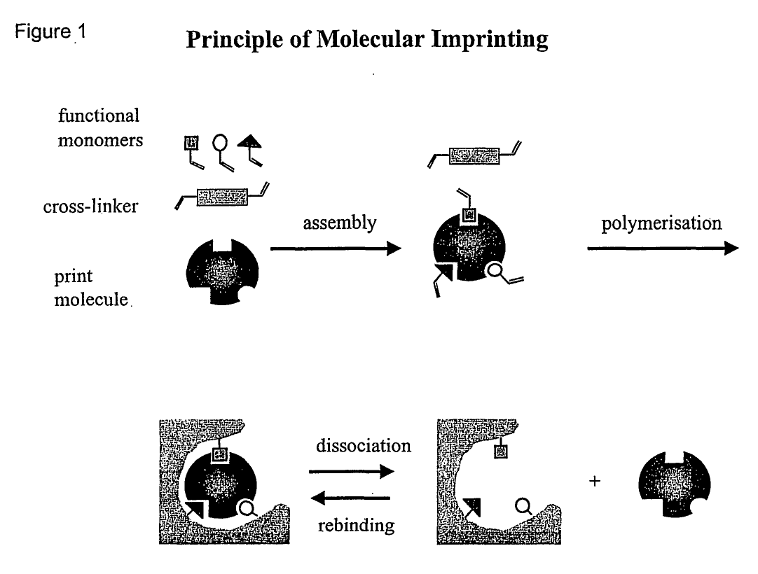

FIG. 2 depicts schematically molecular imprinting using immobilised templates.

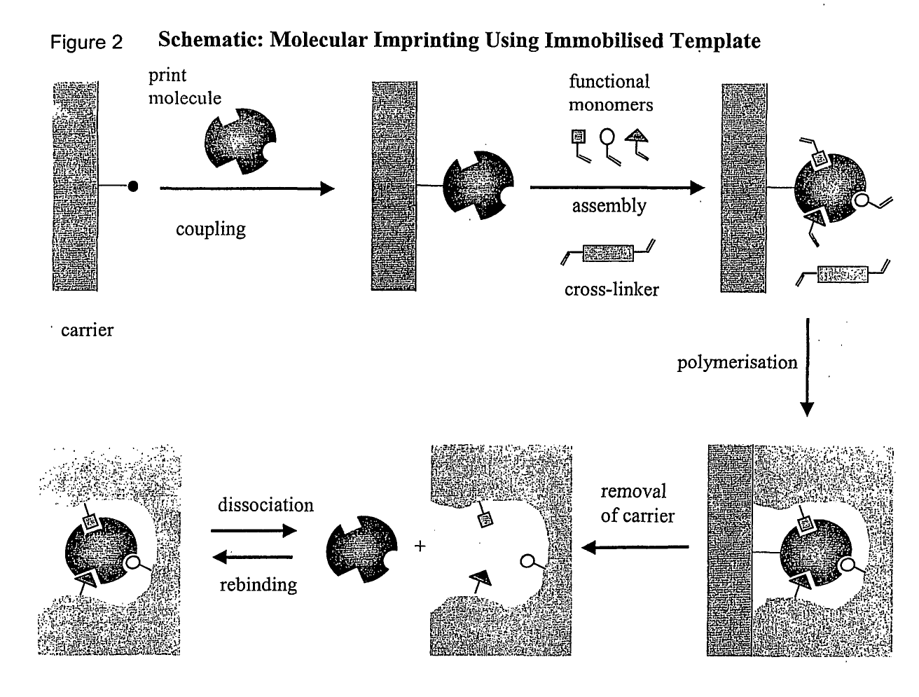

FIG. 3 depicts molecular imprinting using immobilised theophylline.

FIGS. 4 (4A and 4B for protein and gold particle, respectively) depicts schematically the rebinding of labeled analytes onto the MIPs.

### EXAMPLES

### Preparation of Polymers Molecularly Imprinted with Immobilised Templates

### Example 1

### Immobilisation Onto Silica or Glass Surfaces

The template derivatised with a terminal silane functionality is chemically coupled to a silica or a glass surface using standard silanization protocols. Alternatively, the template or an appropriate derivative thereof is chemically coupled onto a functionalised silica or glass surface. Alternatively, the template or an appropriate derivative thereof is allowed to adsorb to a silica or a glass surface. Suitable monomers are then added and polymerised. After completed polymerisation the silica or glass support is removed using aqueous hydrofluoric acid aqueous tetremethylammonium hydroxide or concentrated sodium hydroxide, leaving behind the imprinted polymer. The silica or glass can be in the form of flat substrates, small non-porous particles, or porous beads. In the latter case, the polymer can be synthesised in the pores of the bead.

A more detailed recipe: The coupling of the template to the aminopropyl silica used as the template support was done as follows. Typically 266 mg (1 mmol) 8-carboxypropyl theophylline and 450 μL (3 mmol) DIC were dissolved in 10 mL anhydrous DMF/DCM (1:1, v/v). Then 1 g of dry aminopropyl silica was added and the suspension was shaken on a rocking-table for at least 18 h at room temperature. The coupling reaction was allowed to continue until both Kaiser [15] and TNBS [16] tests were negative, indicating that most aminopropyl groups on the silica surface had reacted. Subsequently, 100 μL (1 mmol) acetic anhydride was added and incubated for another 2 h to acetylate any remaining aminopropyl groups. The silica was then washed on a G4-glass filter funnel with DMF, DCM, and methanol, and dried for 6 h at 45° C. and then in vacuo for a further 6 h.

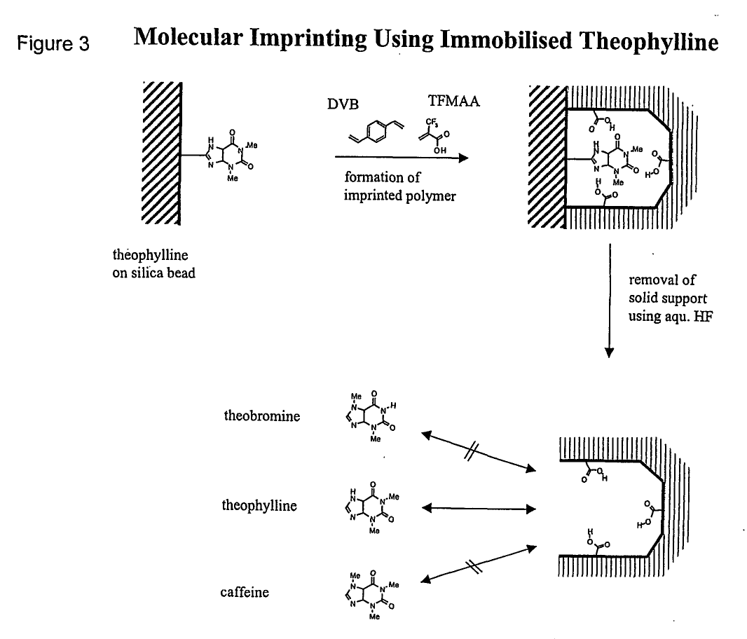

A pre-polymerization mixture consisting of 2.14 mL (12 mmol) DVB, 336 mg (2.4 mmol) TFMAA and 20 mg 2,2′-azobis(2,4-dimethylvaleronitrile) was prepared in a glass vial. According to the pore volume of the silica (ca 0.65 mL g⁻¹ silica), the amount of the mixture required to fill the pores was added to the silica and gently stirred with a stainless steel spatula. The vial was flushed gently with N₂ for 2 min and the mixture was then allowed to polymerise overnight at 45° C. After the polymerisation was completed (this was monitored by polymerising a portion of the pre-polymerization mixture without silica), the silica-polymer composite was gently wet-milled in acetone with a manual mortar and pestle to disintegrate any particle aggregates. The composite was then transferred into a plastic tube with a screw cap, suspended in 2 mL acetone, and cooled in a water/ice bath. To dissolve the silica matrix of the composite, 4 mL aqueous HF (40%) was added portionwise under shaking. The suspension was then allowed to react overnight on a rocking-table at room temperature. The remaining polymer was washed extensively on a G4-glass filter funnel with approx. 2 L of deionized water (containing 20% acetone) until neutrality and finally with 0.25 L methanol. The polymer particles were then dried in an oven at 45° C. for 6 h and in vacuo for a further 6 h (see FIG. 3 for this specific example).

### Example 2

### Immobilisation Onto Latex Beads

The template or a template derivative is chemically coupled or allowed to adsorb onto plain or functionalised latex particles. Suitable monomers are then added and polymerised. After polymerisation, the latex support together with the template is removed for example by extraction with hot toluene, leaving behind the imprinted polymer.

A more detailed recipe: Diaminohexane (DAH) was covalently coupled to carboxylated latex: To 0.5 g cleaned carboxylated latex (corresponding to 0.130 mmol carboxylic acid-groups on the surface) suspended in 5 ml water N-hydoxysuccinimide (NHS) (23 mg, 0.4 mmol) and ethylene-diamine carbodiimide (EDC) (153 mg, 0.65 mmol), each dissolved in 1 ml pure water were added, mixed and allowed to react for 10 min. To this mixture 2 ml of 1 M diamino-hexane solution (2 mmol) was added to give a total volume of approx. 10 ml. The whole mixture was vigorously shaken and allowed to react at least for 2 h at room temp. on a rocking table.

The latex-suspension was then washed successively with 10 ml pure water, 10 ml 1 M NaCl, and three times with 10 ml water again, with centrifugation (18.000 rpm for 10 min.) being performed between each washing stage, and the supernatant being discarded each time. Coupling of the template to the latex: To a suspension of 8-(3-carboxypropyl)-theophylline (173 mg, 0.65 mmol) in water (1 ml) NHS (92 mg, 0.8 mmol) and EDC (613 mg, 3.2 mmol) each dissolved in 1 ml water were added. The DAH-modified latex-suspension was added and mixed, the pH was adjusted to 11 with 1 M NaOH, and then mixed on the vortex for at least 10 min, followed by incubation on a rocking table for 2 h. The coupling reaction was monitored by detection of free primary amino-groups on the latex using the TNBS test [16]. The coupling was performed until no free amino-groups were detectable. The latex-suspension was then washed as described above.

Removal of the water: The latex-suspension was first diluted 1:20 (5 ml+95 ml) with pure water to a final concentration of 0.5% v/v and then sonicated in a 500 ml roundbottom glass flask for at least 30 min. to disintegrate particle agglomeration. This diluted suspension was cooled (−78 C) and lyophilized. Production of the imprinted polymer: The initiator DMPAP (55 mg, 0.2 mmol) was dissolved in the polymer monomers MAA (200 μl, 2.2 mmol) and TRIM (5000 μl, 15.7 mmol) by sonication and then added to the dried latex which was transferred into a screw-cap glasstube. The latex was mixed in the polymer solution, sparged with N₂ for 5 min to remove O₂ and irradiated under an UV-lamp at 366 nm at 0° C. over night to obtain a solid and hard bulk-polymer.

Polymers obtained after completed polymerisation were first manually broken into small pieces and then ground in a mechanical mortar (Retsch, Germany). After grinding the particles were wet sieved with acetone through a 25 μm mesh sieve (Retsch). The fine particles were then removed by sedimentation in acetone.

To dissolve and to remove the incorporated latex from the polymer, the particles were treated with toluene under hot reflux (approx. 100° C.) under stirring. After latex-removal the particles were washed twice with 30 ml acetone and dried in vacuo over night.

### Example 3

### Immobilisation Onto Chitosan

The template or a template derivative is chemically coupled or allowed to adsorb onto plain or activated chitosan surface. Suitable monomers are then added and polymerised. After polymerisation, the chitosan support is removed together with the template for example by extraction with strong acid or base, leaving behind the imprinted polymer.

### Example 4

### Immobilisation Onto Agarose

The template or a template derivative is chemically coupled onto a plain or activated agarose surface (activation may be done by tresyl activation). Suitable monomers are then added and polymerised. After polymerisation, the agarose support and the template are removed by e.g. extraction with hot solvent, leaving behind the imprinted polymer.

### Example 5

### Immobilisation Onto Gold

The template or a template derivative is allowed to adsorb or chemically coupled onto a gold surface. Adsorption can be done directly onto the gold surface, or onto a self-assembled monolayer preformed on the gold surface. Chemical coupling can be done directly onto the gold surface using a thiol-functionalised template derivative, or onto a functionalised self-assembled monolayer preformed on the gold surface. Suitable monomers are then added and polymerised. After polymerisation, the gold support is removed, leaving behind the imprinted polymers.

### Example 6

### Coupling to Polyethylene Glycol (PEG)

The template or a template derivative is coupled to soluble, e.g. terminally functionalised PEG. Suitable monomers are then added and polymerised. After polymerisation the PEG support is removed by extraction using hot water, leaving behind the imprinted polymer.

A more detailed recipe: To a screw-capped glass test tube PEG-bis-theophylline MW 4500 g/mol (250 mg, corresponding to 0.15 mmol theophylline, for the imprinted polymer) or PEG (average FW=5000 g/mol, for the blank polymer) is added. A pre-polymerisation mixture consisting of AIBN (30 mg, 0.2 mmol), EDMA (2265 μl, 12 mmol) and MAA (205 μl, 2.4 mmol) was prepared. To each of the PEGs (PEG-bis-theophylline and plain PEG) 2.9 ml of the pre-polymerisation mixture was added and mixed. After polymerisation, the PEG within the polymer was removed using hot water under reflux. The polymer was then washed with acetone and dried in vacuo leaving behind the imprinted polymer.

Applications of Polymers Molecularly Imprinted with an Immobilised Template

### Example 7

### Enzyme-Linked Molecularly Imprinted Sorbent Assay

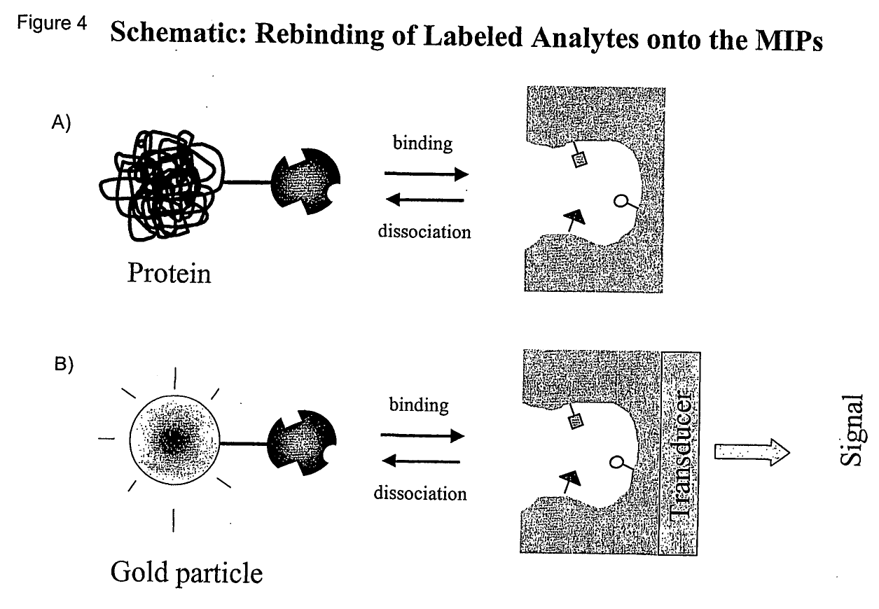

A MIP prepared with an immobilised template is used in a competitive ELISA-type assay, where the target is tagged with an enzyme for detection (FIG. 4A). The binding sites being, inter alia, situated on the surface of the MIP, they can be accessed by a tracer consisting of the target labelled with a comparatively large entity such as an enzyme. In the presence of unlabeled target, some of the tracer is displaced from the polymer. After washing, the remaining tracer is quantified by the enzymatic reaction. This allows a calibration curve for the unlabeled target to be recorded.

### Example 8

### Competitive Acoustic Sensor Using the Target Tagged with Colloidal Gold

A MIP prepared with an immobilised template is used as the recognition element in an acoustic sensor (quartz crystal microbalance, surface acoustic wave sensor), which measures a mass accumulation at or release of accumulated mass from the sensor surface. The binding sites being situated on the surface of the MIP, they can be accessed by the target labelled with colloidal gold particles. In the presence of unlabeled target, some of the gold-labelled targets are displaced from the polymer. After washing, the remaining gold-labelled targets are quantified (FIG. 4B). This allows a calibration curve for the unlabeled target to be recorded. Thereby the increased mass of the target due to the gold label considerably improves the sensitivity of the sensor and lowers the detection limit.

### Example 9

### Use of MIPs Prepared with Immobilised Templates as Separation Materials

A MIP prepared with an immobilised template is used as separation material in chromatography mode as the stationary phase or as a soluble selector. The binding sites are situated on the surface of the pores of the MIP and the pores of the MIP are uniform, monosized and well defined (furthermore, the porosity can be controlled by choosing an appropriate silica template). The binding sites can be accessed easily by the template or a template derivative or a template which is labelled or coupled to another entity. Due to the not hindered accessibility of the binding sites the on-off-kinetics are very fast and the separation takes place at a high performance. Especially the chiral separation performance of enantiomers is highly improved as compared to ‘classical’ MIP systems.

### Example 10

### Use of MIPs Prepared with Immobilised Templates as Specific Chemical Catalysts

A MIP prepared with an immobilised template is used as a nano-cavity for the specific catalysis of desired reactions. The immobilised template may either be a transition state analogue, a substrate or product analogue of one or more entities of the reaction or a shape-forming 10 template, which predetermines the reaction of certain substrates. Such novel catalytic active MIPs have a more enzyme-like behaviour, because the entrance site of the substrate is oriented and is easily accessible and the catalytic active site in the MIP is more uniform.

References

1. Mosbach, K. and Ramström, O. (1996) The emerging technique of molecular imprinting and its future impact on Biotechnology. Bio/Technology 14: 163-170.

2. Vlatakis, G., Andersson, L. I., Muller, R. and Mosbach, K. (1993) Drug assay using antibody mimics made by molecular imprinting. Nature 361: 645-647.

3. Ramström, O., Ye, L. and Mosbach, K. (1996) Artificial antibodies to corticosteroids prepared by molecular imprinting. Chemistry & Biology 3: 471-477.

4. Fischer, L., Müller, R., Ekberg, B., and Mosbach, K. (1991) Direct enantioseparation of β-adrenergic blockers using a chiral stationary phase prepared by molecular imprinting. J. Am. Chem. Soc. 113: 9358-9360.

5. Andersson, L. I. (1996) Application of molecular imprinting to the development of aqueous buffer and organic solvent based radioligand binding assays for (S)-propanolol. Anal. Chem. 68:111-117.

6. Hedborg, E., Winquist,.F., Lundström, I., Andersson, L. I. and Mosbach, K. (1993) Some studies of molecularly imprinted polymer membrane in combination with field-effect devices. Sensors Actuators A 37-38: 796-799.

7. Kriz, D., Ramström, O., Svensson, A. and Mosbach, K. (1995) Introducing biomimetic sensors based on molecularly imprinted polymers as recognition elements. Anal. Chem. 67: 2142-2144.

8. Silke Kröger, Anthony Turner, Klaus Mosbach and Karsten Haupt (1999) Imprinted polymer-based sensor system using differential pulse voltammetry on screen-printed electrodes. Anal. Chem. 71: 3698-3702.

9. Muldoon, M. T. and Stanker, L. H. (1995) Polymer synthesis and characterization of a molecularly imprinted sorbent assay for atrazine. J. Agric. Food Chem. 43: 1424-1427.

Siemann, M., Andersson, L. I. and Mosbach, K. (1996) Selective recognition of the herbicide atrazine by non-covalent molecular imprinted polymers. J. Agric. Food Chem. 44: 141-145.

10. Andersson, L. I., Müller, R. and Mosbach, K. (1996) Molecular imprinting of the endogenous neuropeptide Leu⁵-enkephalin and some derivatives thereof. Macromol. Rapid Commun. 17: 65-71.

11. Wulff, G. 1995. Molecular Imprinting in Cross-Linked Materials with Aid of Molecular Templates—A way towards Artificial Antibodies. Angew. Chem. Int. Ed. Engl. 34: 1812-1832.

12. Ramström, O., L I, Andersson, and K. Mosbach. 1993. Recognition Sites Incorporating Both Pyridinyl and Carboxy Functionalities Prepared by Molecular Imprinting. J. Org. Chem. 58:7562-7564.

13. Whitecombe, M J., M E. Rodriguez, P. Villar, and E N. Vulfson. 1995. A new method for the introduction of recognition site functionality into polymers prepared by molecular imprinting: Synthesis and-characterisation of polymeric receptors for cholesterol. J. Am. Chem. Soc. 117(27):7105-7111.

14. E. Kaiser, R. L. Colescott, C. D. Bossinger, P. I. Cook (1970) Color test for the detection of free terminal amino groups in the solid-phase synthesis of peptides. Anal. Biochem. 34:595-598.

15. W. S. Hancock, J. E. Battersby, (1976) A new micro-test for the detection of incomplete coupling reactions in solid-phase peptide synthesis using 2,4,6-trinitrobenzenesulfonic acid. Anal. Biochem. 71, 260-264.

1. A molecularly imprinted polymer comprising tailor-made recognition sites for a target, in which all said recognition sites are specific binding sites which are freely accessible, uniformly oriented in a defined way and located directly on the surface of the polymer and/or of pores in the polymer, obtainable by polymerising functional monomers and, optionally, cross-linkers, optionally in a reaction solvent, in the presence of at least one template different from the target and immobilised on a support material in a polymerisation process, whereby non-covalent or covalent interactions are formed between said functional monomers and said immobilised template(s), and removing said template(s) and said support material from the molecularly imprinted polymer.

2. A molecularly imprinted polymer according to claim 1, wherein said immobilised template being a shape-forming template.

3. A molecularly imprinted polymer according to claim 1, wherein said immobilised template being a transition state analogue of a reaction or a structural analogue of one or more entities of a reaction.

4. A molecularly imprinted polymer according to any one of claims 1-3, wherein said target is chosen from the group comprising a pesticide, drug, hormone, enzyme, antibody, receptor, nucleic acid, virus, cell, tissue and any other material including proteins.

5. A method for preparing a molecularly imprinted polymer comprising tailor-made recognition sites for a target, characterised by:
polymerising functional monomers and, optionally, cross-linkers, optionally in a reaction solvent, in the presence of at least one template different form the target which is immobilised on a support material in a polymerisation process, whereby non-covalent or covalent interactions are formed between said functional monomers and said immobilised template(s), and
removing said template(s) and said support material from the molecularly imprinted polymer.

6. A method according to claim 5, wherein said immobilised template being a shape-forming template.

7. A method according to claim 5, wherein said immobilised template being a transition state analogue of a reaction or a structural analogue of one or more entities of a reaction.

8. A method according to any one of claims 5-7, wherein said target is chosen from the group comprising a pesticide, drug, hormone, enzyme, antibody, receptor, nucleic acid, virus, cell, tissue and any other material including proteins.

9. A method according to any one of claims 5-8, wherein said support material is present in an insoluble or a colloidal form.

10. A method according to claim 9, wherein said support material is selected from the group comprising of glass, silica, latex beads, polysaccharides, chitosan, ceramics, gold, agarose, other organic or inorganic macromolecular and polymeric materials, and derivatives thereof.

11. A method according to any one of claims 5-8, wherein said support material is present in a soluble form.

12. A method according to claim 11, wherein said support material is selected from the group comprising polyethylene glycol, polyvinylalcohol, polyamide, polyester, other organic and inorganic soluble macromolecular and polymeric materials, and derivatives thereof.

13. A method according to any one of claims 5-12, wherein said template(s) is/are immobilised to said support material by oriented immobilisation, resulting in a uniform orientation of the binding sites.

14. A method according to any one of claims 5-13, wherein said template(s) is/are immobilised to said support material by chemical or physical adsorption.

15. A method according to any one of claims 5-13, wherein said template(s) is/are immobilised to said support material by chemical coupling.

16. A method according to any one of claims 5-15, wherein said template(s) is/are immobilised directly onto said support material or adsorbed onto or inserted in to a self-assembled monolayer preformed on said support material.

17. A method according to any one of claims 5-16, wherein said support materials are functionalised, activated or derivatized.

18. A method according to any one of claims 5-17, wherein said removing is performed in two steps, wherein said support material is removed in one step and said template(s) in another step.

19. A method according to any one of claims 5-17, wherein said removing occurs in one step, wherein said support material and immobilised template(s) are removed at the same time.

20. A method according to any one of claims 5-19, wherein said removing of the immobilised support material and the template(s) is by chemical dissolution, solvent extraction, heat, ultrasonication, acid or base extraction, mechanical or other means.

21. A method according to any one of claims 5-20, wherein said functional monomers have the same functionalities.

22. A method according to any one of claims 5-20, wherein said functional monomers have different functionalities.

23. A method according to any one of claims 5-22, wherein said polymerisation process is a free-radical polymerisation process, an ionic polymerisation process, a co-ordination polymerisation process, a step growth polymerisation process, a living polymerisation or another polymerisation process.

24. A method according to any one of claims 5-23, wherein said polymerisation process is initiated by heat, UV radiation, γ radiation, visible light and/or chemically.

25. Use of the molecularly imprinted polymer as defined in any one of claims 1-4, or prepared according to any one of claims 5-24, as an artificial receptor in applications based on specific recognition and binding.

26. Use of the molecularly imprinted polymer as defined in any one of claims 1-4, or prepared according to any one of claims 5-24, as recognition element in competitive or direct immunoassay-like binding assays.

27. Use of the molecularly imprinted polymer as defined in claim 26, for recognising a pesticide, drug, hormone, enzyme, antibody, receptor, nucleic acid, virus, cell, tissue and any other material including proteins.

28. Use of the molecularly imprinted polymer as defined in any one of claims 1-4, or prepared according to any one of claims 5-24, as tailor-made separation and/or extraction materials.

29. Use of the molecularly imprinted polymer as defined in any one of claims 1-4, or prepared according to any one of claims 5-24, as enzyme-like or chemical catalysts in chemical synthesis or as solid-phase extraction materials for assisted synthesis.

30. Use of the molecularly imprinted polymer as defined in any one of claims 1-4, or prepared according to any one of claims 5-24, as stationary phase or soluble selector in capillary electrophoresis, capillary electro-chromatography or HPLC analysis.

31. Use of the molecularly imprinted polymer as defined in any one of claims 1-4, or prepared according to any one of claims 5-24, as recognition element in a chemical sensor or biosensor.

32. Use according to any one of claims 25-31, wherein said target is tagged with a marker.

33. Use according to claim 32, wherein said marker is an enzyme, a fluorescent, electrochemical, electroluminescent or magnetic marker, a radioisotope, a dye or a colloidal gold particle.
